(19) United States
(12) Patent Application Publication
(10) Pub. No.: US 2005/0075674 A1
(43) Pub. Date: Apr. 7, 2005

(54) Extra-systolic stimulation therapy delivery and sensing via different electrode sets

(76) Inventors: Glenn C. Zillmer, Hudson, WI (US); D. Curtis Deno, Andover, MN (US); Nirav V. Sheth, Coon Rapids, MN (US)

Correspondence Address: MEDTRONIC, INC., 710 MEDTRONIC PARKWAY NE, MS-LC340, MINNEAPOLIS, MN, 55432-5604, US

(21) Appl. No.: 10/680,493

(22) Filed: Oct. 7, 2003

(51) Int. Cl. A61N 1/362

(52) U.S. Cl. 607/9

(57) ABSTRACT

Techniques for delivering ESS to a heart of a patient are disclosed. An implantable medical device delivers ESS stimulation, and in some embodiments pacing stimulation, to a chamber of the heart via a first electrode set. The implantable medical device senses electrical activity within the chamber via a second set of electrodes. In some embodiments, the implantable medical device is able to apply a shorter blanking interval than is typical in the pacing art to a sense amplifier coupled to the second set of electrodes, allowing the implantable medical device to better detect arrhythmias and evoked responses. A variety of electrodes may be used in conjunction with the present invention; including without limitation, tip, ring, coil, can-based, endocardial, epicardial, pericardial, cardiac vein-based, subcutaneous, and/or surface electrodes.

### CROSS-REFERENCE TO RELATED APPLICATIONS

The present patent application hereby cross-references and incorporates by reference the entire contents of the following applications, each of which is filed on even date herewith: non-provisional U.S. application Ser. No. 10/xxx,xxx (Atty. Dkt. P-11155.00) entitled, “REFRACTORY PERIOD TRACKING AND ARRHYTHMIA DETECTION,” non-provisional U.S. application Ser. No. 10/xxx,xxx (Atty. Dkt. P-11548.00), entitled, “METHOD AND APPARATUS FOR CONTROLLING EXTRA-SYSTOLIC STIMULATION (ESS) THERAPY USING ISCHEMIA DETECTION,” non-provisional U.S. application Ser. No. 10/xxx,xxx (Atty. Dkt. P-11354) entitled, “METHOD AND APPARATUS FOR OPTIMIZATION AND ASSESSMENT OF RESPONSE TO EXTRA-SYSTOLIC STIMULATION (ESS) THERAPY,” non-provisional U.S. application Ser. No. 10/xxx,xxx (Atty. Dkt. P-11193) entitled, “MULTIPLE PACING OUTPUT CHANNELS,” provisional U.S. application Ser. No. 60/xxx,xxx (Atty. Dkt. P-11438.00) entitled, “CARDIAC PACING MODALITY HAVING IMPROVED BLANKING, TIMING, AND THERAPY DELIVERY METHODS FOR EXTRA-SYSTOLIC STIMULATION PACING THERAPY,” and provisional U.S. application Ser. No. 60/xxx,xxx (Atty. Dkt. P-11359.00) entitled, “SECURE AND EFFICACIOUS THERAPY DELIVERY FOR AN EXTRA-SYSTOLIC STIMULATION PACING ENGINE.”

### TECHNICAL FIELD

The invention relates to medical devices, and more particularly, to medical devices for delivery of extra-systolic stimulation therapy.

### BACKGROUND

Extra-systolic stimulation (ESS) therapy involves the delivery of an extra-systolic pacing pulse to a chamber of the heart an extra-systolic interval (ESI) after a paced or spontaneous depolarization of that chamber. For this reason, ESS therapy is sometimes referred to as paired, coupled, or bi-geminal pacing. The extra-systolic pulse is applied after the refractory period that follows the first paced or spontaneous depolarization, and results in a subsequent electrical depolarization of the chamber without an attendant myocardial contraction. Because it results in an electrical depolarization, the extra-systolic pulse may be referred to as an “excitatory” cardiac stimulation pulse.

The second depolarization of the chamber effectively slows the heart rate from its spontaneous rhythm, allowing a greater time for filling of the chamber. Further, the second depolarization of the chamber causes an augmentation of contractile force of the chamber during the heart cycle following the one in which the extra-systolic pulse is applied. Increased filling and contractile force augmentation causes increased stroke volume and can under certain circumstances lead to increased cardiac output, particularly when ESS therapy is delivered to one or more of the ventricles of the heart. For this reason, ESS therapy has been proposed as a therapy for patients with congestive heart failure (CHF) and/or left ventricular dysfunction (LVD).

In general medical devices used to deliver ESS therapy, such as implantable pacemakers, include sense amplifiers coupled to electrodes that detect cardiac depolarizations. The medical devices may, for example, control the timing of delivery of pacing and ESS pulses, confirm that pacing and ESS pulses captured the heart, and detect arrhythmias based on detected depolarizations. However, the myocardial tissue proximate to electrodes typically become polarized temporarily for a period of time subsequent to delivery of an ESS therapy stimulation pulse via the electrodes, which can lead to saturation of the sense amplifier coupled to the electrodes until the polarization dissipates. Often, the sense amplifier is blanked, e.g., decoupled from the electrodes, for a period of time, e.g., a blanking period, following delivery of a stimulation pulse to avoid saturation of the sense amplifier.

Whether saturated or blanked, the sense amplifier is unable to detect any intrinsic cardiac activity for a period of time following delivery of a stimulation pulse via the electrodes to which it is coupled. Consequently, where a medical device delivers both a pacing pulse and one or more ESS pulses during a single cardiac cycle, the sense amplifier will be unable to detect intrinsic activity of the heart for a significant portion of that cardiac cycle. This, in turn, may make it difficult for the medical device to, for example, detect potentially deadly arrhythmias.

### SUMMARY

In general, the present invention is directed to techniques for delivering ESS to a heart of a patient. An implantable medical device delivers ESS stimulation, and in some embodiments pacing stimulation, to a chamber of the heart via a first electrode set. The implantable medical device senses electrical activity within the chamber via a second set of electrodes. In some embodiments, the implantable medical device is able to apply a shorter blanking interval than is typical in the pacing art to a sense amplifier coupled to the second set of electrodes, allowing the implantable medical device to better detect cardiac arrhythmias, intrinsic activity and evoked responses.

In some embodiments, the first set of electrodes includes a bipolar electrode pair carried on a lead that extends into the chamber. In various embodiments, the second set of electrodes includes bipolar electrode pairs disposed within, about or on (i.e., epicardial) the heart or other chambers of the heart, unipolar combinations of such electrodes and/or at least one electrode integrated with the housing of the implantable medical device, one or more coil electrodes, a tip electrode, a ring electrode of the first set of electrodes, a subcutaneous electrode array, a surface electrode, an endocardial electrode, an epicardial electrode, an pericardial electrode, a cardiac vein-based electrode, or any combination of these electrodes. Some embodiments include a second lead that extends into the chamber, and carries a second set of electrodes for sensing electrical activity within the chamber.

In one embodiment, the invention is directed to a method in which excitatory extra-systolic electrical stimulation is delivered to a chamber of a heart of a patient via a first set of electrodes, and electrical activity within the chamber is sensed via a second set of electrodes.

In another embodiment, the invention is directed to a medical device system comprising a medical device coupled to first and second sets of electrodes. The medical device delivers excitatory extra-systolic electrical stimulation to a chamber of a heart of a patient via the first set of electrodes and senses electrical activity within the chamber via the second set of electrodes.

In another embodiment, the invention is directed to a medical device system comprising an implantable pacemaker implanted within a patient, and first and second leads that extend from the pacemaker to positions within a chamber of a heart of the patient. The system further includes a first pair of electrodes that is located proximate to a distal end of the first lead, and a second pair of electrodes that is located proximate to a distal end of the second lead. The pacemaker delivers excitatory extra-systolic stimulation to the chamber via the first pair of electrodes, and senses electrical activity within the chamber via the second pair of electrodes.

The details of one or more embodiments of the invention are set forth in the accompanying drawings and the description below. Other features, objects, and advantages of the invention will be apparent from the description and drawings, and from the claims.

### BRIEF DESCRIPTION OF DRAWINGS

FIG. 1 is a conceptual diagram illustrating an exemplary medical device system that includes an implantable medical device that delivers extra-systolic stimulation therapy implanted within a patient.

FIG. 2 is a conceptual diagram further illustrating the implantable medical device of FIG. 1 and the heart of the patient.

FIG. 3 is a functional block diagram of the implantable medical device of FIG. 1.

FIG. 4 is a timing diagram illustrating exemplary blanking intervals applied by the implantable medical device of FIG. 1 according to the invention.

FIG. 5 is a conceptual diagram illustrating another example medical device system according to the invention.

FIG. 6 is a conceptual diagram illustrating another example medical device system according to the invention.

### DETAILED DESCRIPTION

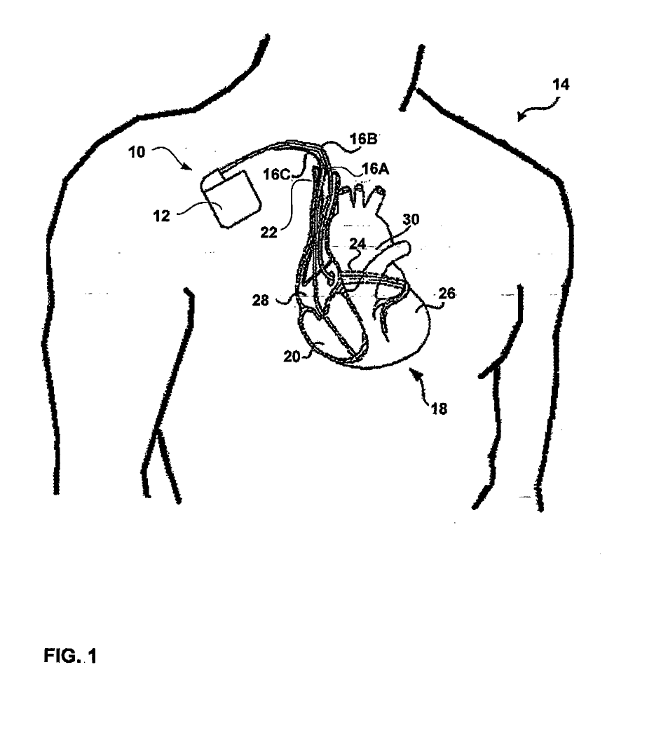

FIG. 1 is a conceptual diagram illustrating an exemplary medical device system 10, which includes an implantable medical device (IMD) 12 implanted within a patient 14. IMD 12 delivers extra-systolic stimulation (ESS) therapy to the heart 18 of patient 14. In the illustrated embodiment, IMD 10 takes the form of a multi-chamber cardiac pacemaker.

System 10 further includes leads 16A,16B,16C (collectively “leads 16”) that are coupled to IMD 12 and extend into the heart 18 of patient 14. More particularly, right ventricular (RV) lead 16A extends through one or more veins (not shown), the superior vena cava (SVC) 22, and right atrium 28, and into right ventricle 20. Left ventricular (LV) coronary sinus lead 16B extends through the veins, the SVC 22, right atrium 28, and into the coronary sinus 24 to a point adjacent to the free wall of left ventricle 26 of heart 18. Right atrial (RA) lead 16C extends through the veins and SVC 22, and into the right atrium 28 of heart 18.

Each of leads 16 includes electrodes (not shown in FIG. 1). IMD 12 delivers ESS to one or more of chambers 20,26,28 via electrodes carried by one or more of leads 16. In some embodiments, IMD 12 also delivers pacing stimulation, i.e., stimulation intended to cause a depolarization and contraction of heart 18, to one or more of chambers 20,26,28 via electrodes carried by one or more of leads 16. In exemplary embodiments, IMD 12 delivers ESS and pacing stimulation in the form of pulses, which in various embodiments have a single phase, are biphasic, or are multiphasic. The electrodes located on leads 16 are unipolar or bipolar, as is well known in the art.

As will be described in greater detail below, IMD 12 senses electrical activity within chambers 20,26,28 via a different set of the electrodes carried on leads 16 than is used to deliver ESS stimulation to that chamber. In other words, when IMD 12 delivers ESS stimulation to one of chambers 20,26,28 via a first set of the electrodes carried on leads 16, IMD 12 senses electrical activity within that chamber via a second set of the electrodes carried on leads 16. In some embodiments in which IMD 12 also delivers pacing stimulation, IMD 12 may deliver the pacing stimulation to the chamber via the first set of the electrodes. In exemplary embodiments, the first and second sets of electrodes are first and second pairs of electrodes.

In general, the electrodes of the second set of electrodes are not immediately proximate to the site at which IMD 12 delivers pacing and ESS stimulation via the first set of electrodes. Consequently, impairment of the ability of IMD 12 to detect depolarizations of heart 18 via the second set of electrodes due to polarization of the myocardium resulting from delivery of stimulation via the first set of electrodes will not be as great as that experienced by convention IMDs that sense electrical activity of heart 18 via the same set of electrodes used to deliver stimulation. In exemplary embodiments, IMD 12 applies a shorter blanking interval subsequent to delivery via the first set of electrodes when sensing via the second set of electrodes than is typically applied by conventional IMDs that sense via the same set of electrodes used for stimulation delivery.

By sensing electrical activity within a chamber via a second set of electrodes IMD 12 is able to detect depolarizations within the chamber that might have been missed by conventional IMDs due to myocardial polarization and longer blanking intervals. By detecting these depolarizations, IMD 12 can more effectively detect potentially lethal cardiac arrhythmias, and can also detect evoked responses resulting from delivery of stimulation via the first set of electrodes. In exemplary embodiments, IMD 12 provides anti-tachycardia pacing, cardioversion, and/or defibrillation therapies to heart 18 in response to detection of an arrhythmia via electrodes carried on leads 16. In some embodiments, IMD 12 detects evoked responses subsequent to delivery of pacing and ESS stimulation to determine whether the stimulation captured heart 18, and can adjust the intensity and/or timing of the stimulation to maintain or reacquire capture in response to the determination.

The configuration of system 10 illustrated in FIG. 1 is merely exemplary. An IMD 12 according to the invention may be coupled to any number of leads 16 that extend to any position within or on the surface of heart 18. For example, some medical device system embodiments according to the invention include a single lead 16A or 16C that extends into right ventricle 20 or right atrium 28, respectively, or two leads 16A,16C that extend into the right ventricle 20 and right atrium 28, respectively. Some embodiments include leads 16A-C located as illustrated in FIG. 1, and an additional lead 16 located within or proximate to right ventricle 20. Further some embodiments include one or more leads 16 that extend to a position within left atrium 30.

Some embodiments include epicardial leads instead of or in addition to the transvenous leads 16 illustrated in FIG. 1. Further, medical device systems according to the invention need not include an IMD 12 implanted within patient 14, but may instead include an external medical device that delivers stimulation to heart 18. Such an external medical device can deliver pacing and ESS stimulation to heart 18 via percutaneous leads 16 that extend through the skin of patient 14 to a variety of positions within or outside of heart 18, or transcutaneous electrodes placed on the skin of patient 14.

In exemplary embodiments, IMD 12 delivers ESS stimulation in the form of electrical pulses. IMD 12 delivers ESS pulses to one or more of chambers 20, 26 and 28 an extra-systolic interval (ESI) after an intrinsic or paced depolarization of that chamber. In various embodiments, IMD 12 delivers ESS pulses continuously, periodically, in response to user activation, as a function of measured physiological parameters, or the like. Exemplary techniques for delivering and controlling delivery of ESS are described in commonly-assigned U.S. Pat. Nos. 5,213,098 and 6,438,408 and commonly-assigned co-pending non-provisional U.S. patent application Ser. Nos. 10/322,792 (Atty. Dkt. P-9854.00) filed 28 Aug. 2002 (P-9854.00) and 10/426,613 (Atty. Dkt. P-11214.00) filed 29 Apr. 2003 each of which is incorporated herein by reference in its entirety.

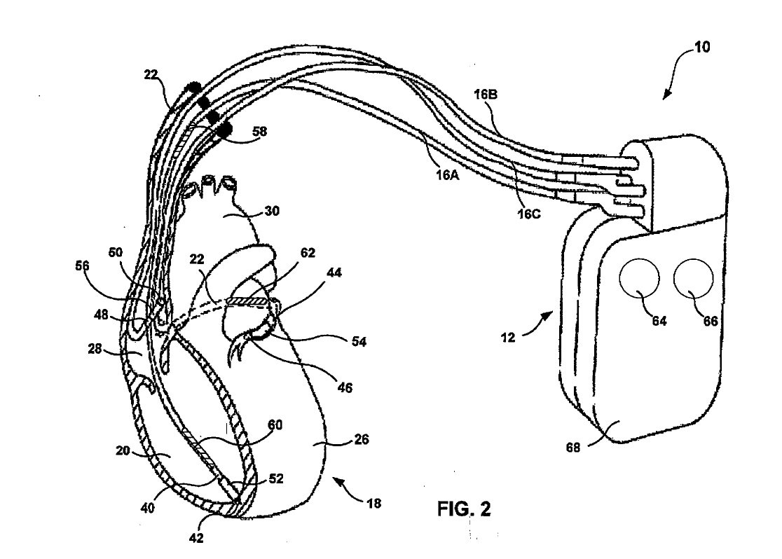

FIG. 2 is a conceptual diagram further illustrating system 10. In some embodiments, each of leads 16 includes an elongated insulative lead body carrying a number of concentric coiled conductors separated from one another by tubular insulative sheaths. Located adjacent distal end of leads 16A, 16B, and 16C are bipolar electrode pairs 40 and 42, 44 and 46, and 48 and 50 respectively. In the illustrated embodiment, electrodes 40,44,48 take the form of ring electrodes, and electrodes 42,46,50 take the form of extendable helix tip electrodes mounted retractably within insulative electrode heads 52,54,56 respectively. Each of the electrodes 40-50 is coupled to one of the coiled conductors within the lead body of its associated lead 16.

In the illustrated embodiment, IMD 10 also includes indifferent housing electrodes 64 and 66, formed integrally with a hermetically sealed housing 68 of IMD 12. In some embodiments, IMD 12 delivers pacing and ESS stimulation to one or more of chambers 20, 26 and 28 via the respective one or more of bipolar electrode pairs 40 and 42, 44 and 46, and 48 and 50. In other embodiments, IMD 12 delivers unipolar pacing and ESS stimulation to one or more of chambers 20,26,28 via the respective one or more of tip electrodes 42,46,50 in combination with one of housing electrodes 64 and 66.

In exemplary embodiments, IMD 12 delivers cardioversion and/or defibrillation therapy to heart 18 via one or more of elongated coil electrodes 58,60,62. In the illustrated embodiment, coil electrodes 58 and 60 are carried on lead 16A, and coil electrode 62 is carried on lead 16B. Coil electrodes 58, 60, and 62 are located in the SVC 22, right ventricle 20, and coronary sinus 24, respectively. Coil electrodes 58-62 are fabricated from platinum, platinum alloy or other materials known to be usable in implantable defibrillation electrodes, and may be about 5 cm in length.

As discussed above, IMD 12 delivers pacing and ESS stimulation to one or more of chambers 20,26,28 via a first set of electrodes, and senses electrical activity within via that chamber via a second set of electrodes. For example, in embodiments where IMD 12 delivers pacing and ESS stimulus to right ventricle 20 via electrodes 40 and 42, IMD 12 may sense electrical activity within right ventricle 20 via any combination of electrodes 44,46,48,50,58,60,62,64,66. In some embodiments, the first and second set of electrodes include one or more common electrodes. For example, in some embodiments where IMD 12 delivers pacing and ESS stimulation to right ventricle 20 via electrodes 40,42, the second set of electrodes can include ring electrode 40.

Again, the configuration of system 10 illustrated in FIG. 2 is merely exemplary. System 10 may include any number of electrodes located on a variety of leads and positioned within or on the surface of heart 18. In some embodiments, for example, SVT coil electrode 58 is carried on lead 16B or 16C. In other embodiments, IMD 12 is not coupled to coil electrodes or does not include housing electrodes. Further, IMD 12 need not deliver pacing stimulation, and can deliver ESS stimulation to any one or more of chambers 20,26,28,30.

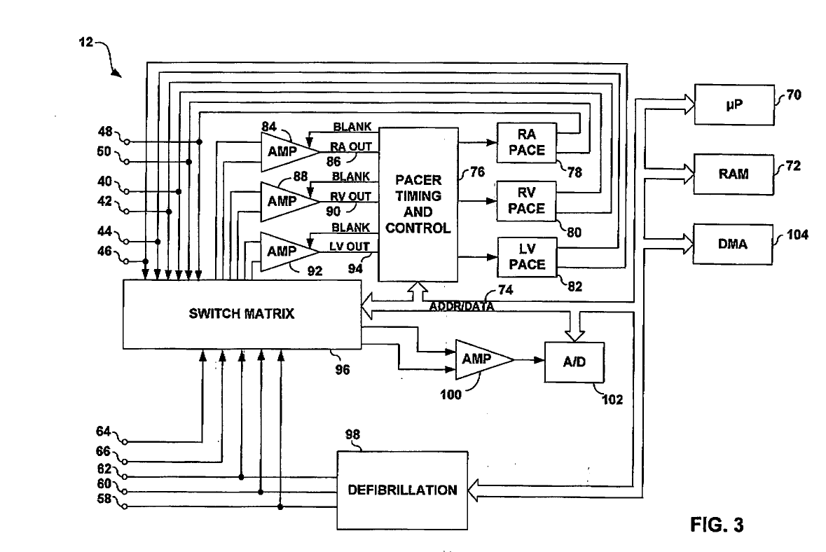

FIG. 3 is a functional block diagram illustrating an exemplary configuration of IMD 12. As shown in FIG. 3, IMD 12 takes the form of a multi-chamber implantable cardioverter-defibrillator (or a pacemaker-cardioverter-defibrillator) having a microprocessor-based architecture. However, this diagram should be taken as exemplary of the type of device in which various embodiments of the present invention may be embodied, and not as limiting. For example, it is believed that the invention may be practiced in a wide variety of device implementations, including devices that provide ESS stimulation but do not provide pacemaker and/or defibrillator functionality.

IMD 12 includes a microprocessor 70. Microprocessor 70 executes program instructions stored in memory, such as a read-only memory (ROM) (not shown), electrically-erasable programmable ROM (EEPROM) (not shown), and/or random access memory (RAM) 72, which control microprocessor 70 to perform the functions ascribed to microprocessor 70 herein. Microprocessor 70 is coupled to, e.g., to communicates with and/or controls, various other components of IMD 12 via an address/data bus 74.

IMD 12 senses electrical activity within heart 18, delivers ESS stimulation to heart 18, and, in some embodiments, delivers pacing stimulation to heart 18. In exemplary embodiments, pacer timing/control circuitry 76 controls delivery of ESS and pacing pulses by one or more of output circuits 78-82 via electrodes 40-50. Specifically, output circuit 78 is coupled to electrodes 48,50 to deliver ESS and/or pacing pulses to right atrium 28, output circuit 80 is coupled to electrodes 40 and 42 to deliver ESS and/or pacing pulses to right ventricle 20, and output circuit 82 is coupled to electrode 44,46 to deliver ESS and/or pacing pulses to left ventricle 26. Output circuits 78-82 include known circuitry for storage and delivery of energy in the form of pulses, such as switches, capacitors, and the like.

Pacer timing/control circuitry 76 includes programmable digital counters that control the timing of delivery of ESS pulses, the values of which are set based on information received from microprocessor 70 via data bus 74. In exemplary embodiments, circuitry 76 controls the interval between a paced or spontaneous depolarization and delivery of an extra-systolic pulse to heart 16 for delivery of ESS, i.e., the extra-systolic interval (ESI). Circuitry 76 also preferably controls escape intervals associated with pacing, such as atrial and/or ventricular escape intervals associated with a selected mode of pacing. In some embodiments, IMD 12 delivers a cardiac resynchronization therapy (CRT), and circuitry 76 controls a V-V interval for delivery of bi-ventricular pacing.

Pacer/timing control circuitry 76 resets interval counters upon detection of R-waves or P-waves, or generation of pacing pulses, and thereby controls the basic timing of ESS and cardiac pacing functions. Intervals defined by pacing circuitry 76 may also include refractory periods during which sensed R-waves and P-waves are ineffective to restart timing of escape intervals, and the pulse widths of the pacing pulses. The durations of these intervals are determined by microprocessor 70 in response to data stored in RAM 72, and are communicated to circuitry 76 via address/data bus 74. The amplitude of the ESS and/or pacing pulses, e.g., the energy stored in capacitors of output circuits 78-82, is also determined by circuitry 76 under control of microprocessor 70.

Microprocessor 70 operates as an interrupt driven device, and is responsive to interrupts from pacer timing/control circuitry 76 corresponding to the occurrence of sensed P-waves and R-waves and corresponding to the generation of cardiac pacing pulses. Circuitry 76 provides such interrupts to microprocessor 70 via data/address bus 74. Any necessary mathematical calculations to be performed by microprocessor 70 and any updating of the values or intervals controlled by pacer timing/control circuitry 76 take place following such interrupts.

IMD 12 senses electrical activity within heart 18 via sense amplifiers 84,88,92, which sense electrical activity within right atrium 28, right ventricle 20, and left ventricle 26, respectively. As discussed above, IMD 12 senses electrical activity within a chamber of heart 18 via a different set of electrodes than is used to deliver ESS and pacing stimulation to the chamber. In the illustrated embodiment, any of electrodes 40-50 and 58-64 may be selectively coupled to one or more of sense amplifiers 84,88,92 via a switch matrix 96 in order to couple second sets of electrodes to the sense amplifiers 84,88,92. The electrode/amplifier assignments can be provided to switch matrix 96 and varied as needed by microprocessor 70 via address/data bus 74, and may be programmed or altered by a user via device telemetry techniques known in the art.

Sense amplifiers 84, 88 and 92 take the form of an automatic gain controlled amplifiers providing an adjustable sensing threshold as a function of the measured P-wave or R-wave amplitude. Sense amplifiers 84,88,92 generate signals on RA out line 86, RV out line 88 and LV out line 92, respectively, whenever the signal sensed between the electrodes coupled thereto exceeds the present sensing threshold. Thus, sense amplifiers 84,88,92 are used to detect intrinsic right atrial, right ventricular, and left ventricular depolarizations, e.g., P-waves and R-waves, respectively.

As illustrated in FIG. 3, pacer timing/control circuit 76 applies blanking signals to sense amplifiers 84,88,92 subsequent to delivery of ESS and pacing stimulation. In exemplary embodiments, the blanking signals cause the amplifiers to decouple from their selected electrodes for a blanking interval, as is known in the art. Because amplifiers 84,88,92 are not coupled to electrode pairs 48 and 50, 40 and 42, and 44 and 46, respectively, the blanking intervals applied by circuit 76 may be shorter than those applied by conventional IMDs. As discussed above, the shorter blanking intervals allow the amplifiers to sense depolarizations during a greater portion of each cardiac cycle, allowing IMD 12 to more effectively detect evoked responses and arrhythmias.

The illustrated configuration of IMD 12 is merely exemplary. For example, IMD 12 need not include switch matrix 96, and/or electrodes need not be selectively coupled to sense amplifiers 84,88,92 via switch matrix 96. In some embodiments, one or more of amplifiers 84,88,92 are directly and permanently coupled to a second set of electrodes. Further, although each of sense amplifiers 84,88,92 are illustrated in FIG. 3 as coupled to a second set of electrodes, the invention is not so limited. Rather, in some embodiments, one or more of the sense amplifiers are coupled to the respective one of bipolar electrode pairs 40 and 42, 44 and 46, and 48 and 50.

In some embodiments, IMD 12 detects ventricular and/or atrial tachycardias or fibrillations of heart 18 using tachycardia and fibrillation detection techniques and algorithms known in the art. For example, the presence of a ventricular or atrial tachycardia or fibrillation can be confirmed by detecting a sustained series of short R-R or P-P intervals of an average rate indicative of tachycardia, or an unbroken series of short R-R or P-P intervals. IMD 12 is also capable of delivering one or more anti-tachycardia pacing (ATP) therapies to heart 18, and/or defibrillation or cardioversion pulses to heart 18 via one or more of electrodes 58-62.

Electrodes 58-62 are coupled to defibrillation circuit 98, which delivers defibrillation and/or cardioversion pulses under the control of microprocessor 70. Defibrillation circuit 98 includes energy storage circuits such as capacitors, switches for coupling the storage circuits to electrodes 58-62, and logic for controlling the coupling of the storage circuits to the electrodes to create pulses with desired polarities and shapes. Microprocessor 70 may employ an escape interval counter to control timing of such defibrillation pulses, as well as associated refractory periods. IMD 10 may include defibrillator functionality where patient 12 has a history of tachyarrhythmia, or to address possibility of tachyarrhythmia associated with ESS therapy. In some embodiments, microprocessor 70 analyzes an electrogram signal that represents electrical activity of heart 18 to, for example, detect cardiac arrhythmias. Switch matrix 96 is used to select which of the available electrodes 40-50 and 58-66 are coupled to wide band (0.5-200 Hz) amplifier 100 for use in digital signal analysis. Selection of electrodes is controlled by microprocessor 70 via data/address bus 74, and the selections may be varied as desired. The analog signals derived from the electrodes selected by switch matrix 96 and amplified by amplifier 100 are converted to a multi-bit digital signal by A/D converter 102, and the digital signal is digitally processed by microprocessor 70. In some embodiments, the digital signal is stored in RAM 72 under control of direct memory access circuit (DMA) 104 for later analysis by microprocessor 70.

Although described herein in the context of a microprocessor-based pacemaker embodiment IMD 10, the invention may be embodied in various implantable medical devices that include one or more processors, which may be microprocessors, controllers, digital signal processors (DSPs), field-programmable gate arrays (FPGAs), or other digital logic circuits.

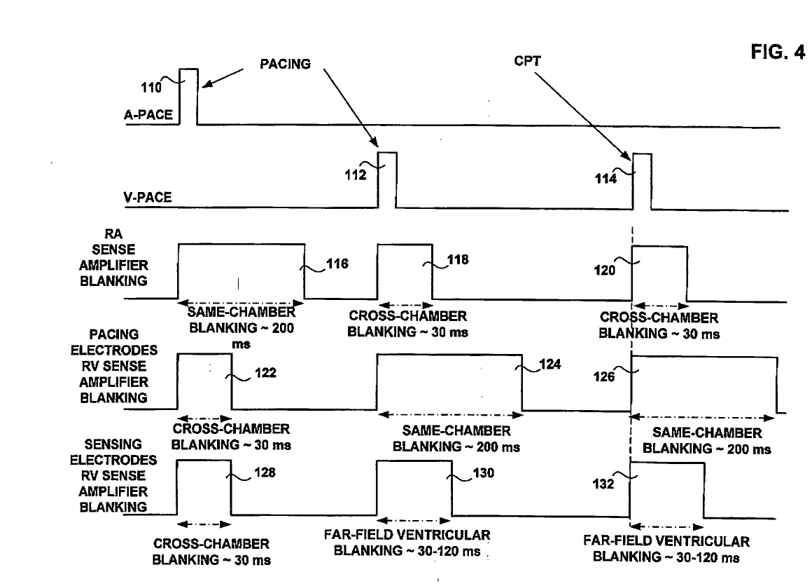

FIG. 4 is a timing diagram illustrating exemplary blanking intervals applied by IMD 12 according to the invention. More specifically, FIG. 4 illustrates blanking intervals applied by IMD 12 during a single cardiac cycle in which pacing pulses 110 and 112 are delivered to right atrium 28 and right ventricle 20, respectively, and a ESS pulse 114 is delivered to right ventricle 20 an ESI after delivery of pacing pulse 112. IMD 12, and more particularly pacer timing/control circuit 76, applies blanking intervals to sense amplifiers 84, 88 and 92 after delivery of pulses 110-114 as illustrated in FIG. 4. Blanking intervals 128-132 applied by IMD 12 are illustrated in comparison with blanking intervals 116-126 typically applied by conventional IMDs that sense electrical activity within right ventricle 20 via electrodes 40 and 42.

As illustrated in FIG. 4, when conventional IMDs deliver pulses via a pair of electrodes that are coupled to a sense amplifier, same-chamber blanking intervals 116,124,126 on the order of 200 milliseconds (ms) are applied to the sense amplifier. When pulses are delivered to another chamber, substantially shorter cross-chamber blanking intervals 118,120,122, on the order of 30 ms, are applied to the sense amplifier. As illustrated in FIG. 4, total blanking of the right ventricular sense amplifier of a conventional IMD can be as great as 430 ms of a signal cardiac cycle. Total blanking times this great can significantly impair detection algorithms for sensing fast ventricular rhythms such as ventricular tachycardia and fibrillations, and the ability of IMD 12 to detect evoked responses in order to perform capture detection functions. The total time that the sense amplifiers of conventional IMDs is blanked is even greater where atrial compensatory pacing pulses (not shown), the functions of which are described in greater detail in the incorporated references listed above, are delivered to the atria in additional to delivery of ESS pulses to the ventricles.

Because IMD 12 senses electrical activity in right ventricle 20 via a second set of electrodes, shorter “far-field” ventricular blanking intervals 130,132 are applied to sense amplifier 80 instead of same-chamber blanking intervals 124,126. Far-field blanking intervals 130,132 can be between 30 and 120 ms, resulting in a total blanking time for the cycle of between 90 and 270 ms. The length of far-field blanking intervals 130,132 can be selected and/or adjusted depending on the purpose for which IMD 12 wishes to detect during a greater portion of the cardiac cycle. For example, shorter blanking intervals on the order of 30 ms may be necessary to detect evoked responses, while longer blanking intervals on the order of 120 ms may be desirable for arrhythmia detection in that counting evoked responses as beats may be avoided.

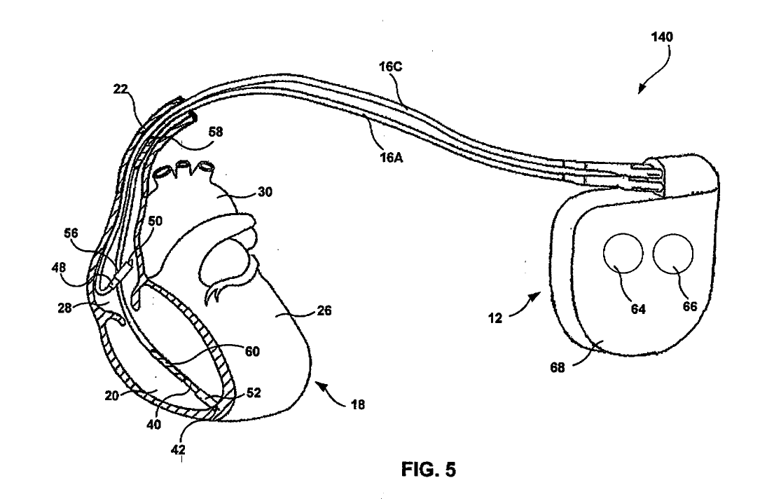

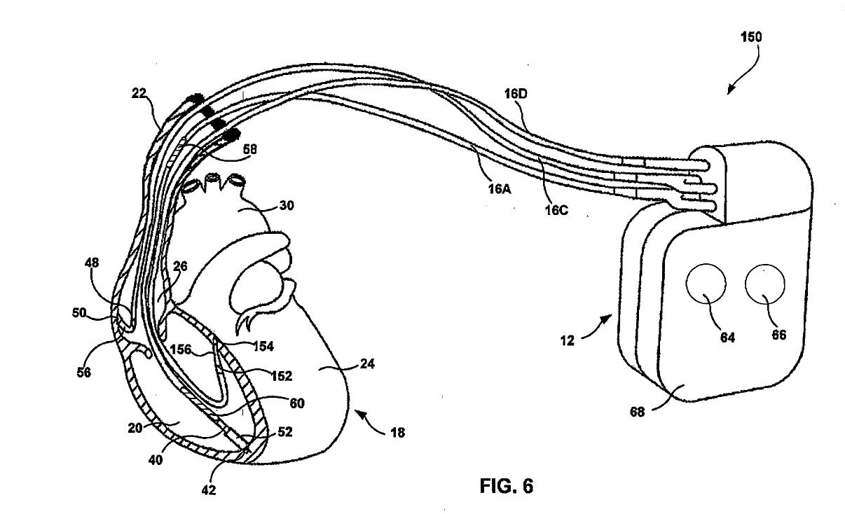

FIGS. 5 and 6 are conceptual diagrams illustrating additional example medical device systems 140,150 according to the invention. In particular, systems 140,150 illustrate alternative configurations of leads 16 that may be employed according to the invention. Medical device 140, for example, includes a single lead 16C that extends to right atrium 28 and a single lead 16A that extends to right ventricle 20 of heart 18. In such embodiments, IMD 12 can deliver ESS pulses to right ventricle 20 via electrodes 40,42, and sense electrical activity within right ventricle via any combination of electrodes 40,48,50,58,60,64,66.

Medical device system 150 illustrated in FIG. 6 includes a single lead 16C that extends to right atrium 28, and two leads 16A,16D that extend to right ventricle 20 of heart 18. Through the provision of two leads within right ventricle 20, IMD 12 of medical system 150 more effectively sense electrical activity within right ventricle 18 without employing the set of electrodes used to deliver ESS and pacing stimulation. Specifically, in exemplary embodiments, IMD 12 delivers stimulation via one of bipolar electrode pairs 40,42 and 152,154, and senses electrical activity via the other pair. Electrodes 152,154 take the form or ring and tip electrodes, respectively, and tip electrode 154 is an extendable helix tip electrode mounted retractably within insulative electrode head 156.

In the illustrated embodiment, lead 16A extends to the apex of right ventricle 20 and lead 16D extends to the septum of right ventricle 20. In some embodiments, lead 16D alternatively extends to the ventricular outflow tract (VOT) of right ventricle 20. In exemplary embodiments, IMD 12 senses electrical activity via electrodes 40,42 at the customary apical location, which may improve the ability of IMD 12 to discern arrhythmias using common arrhythmia detection techniques, and delivers ESS and pacing pulses electrodes 152,154 to an alternative site, such as the septal wall or VOT. Further, delivery of pacing stimulation to a non-apical location, such as the septal wall or VOT, can improve synchronicity of the resulting ventricular contraction.

Various embodiments of the invention have been described. These and other embodiments are within the scope of the following claims. And, as is well known in the field of medical device technology the methods of the present invention may be implemented in any suitable processor-controlled device. Accordingly, said methods embodied as executable instructions for performing the methods may be stored on any computer readable medium. The present invention expressly includes all types of such computer readable media if said methods are stored thereon.

1. A method comprising:
delivering excitatory extra-systolic electrical stimulation to a chamber of a heart of a patient via a first set of electrodes; and
sensing electrical activity of the chamber via a second set of electrodes.

2. The method of claim 1, wherein the electrodes of the first and second sets of electrodes are adapted to be chronically implanted within the patient.

3. The method of claim 2, wherein the electrodes of the first and second sets of electrodes are adapted to be chronically located within the heart.

4. The method of claim 1, wherein the first and second sets of electrodes are first and second pairs of electrodes.

5. The method of claim 1, wherein the first set of electrodes includes a first tip electrode and a first ring electrode, and the second set of electrodes includes at least one of the first ring electrode, a second tip electrode, an second ring electrode, a coil electrode, a can-based electrode, a pericardial electrode, an epicardial electrode, an endocardial electrode, an cardiac vein-based electrode.

6. The method of claim 1, wherein the electrodes of the first and second set of electrodes are operatively coupled to a single medical electrical lead.

7. The method of claim 1, wherein the electrodes of the first set of electrodes are operatively coupled to a first lead, the electrodes of the second set of electrodes are operatively coupled to a second lead, and the first and second leads are adapted to extend into the chamber.

8. The method of claim 1, wherein delivering excitatory extra-systolic electrical stimulation to a chamber comprises delivering excitatory extra-systolic electrical stimulation to a right ventricle of the heart, and sensing electrical activity within the chamber comprises sensing electrical activity within the right ventricle.

9. The method of claim 8, wherein delivering excitatory extra-systolic electrical stimulation to a right ventricle comprises delivering excitatory extra-systolic electrical stimulation to one of a site proximate to an outflow tract of the heart and a ventricular septum of the heart, and wherein sensing electrical activity within the right ventricle comprises sensing electrical activity from a site proximate to an apex of the heart.

10. The method of claim 1, wherein sensing electrical activity within the heart comprises applying a far-field blanking interval to a sense amplifier coupled to the second set of electrodes in response to delivery of excitatory extra-systolic electrical stimulation via the first set of electrodes.

11. The method of claim 10, wherein a length of the far-field blanking interval is less than approximately 300 milliseconds

12. The method of claim 10, wherein the length of the far-field blanking interval is between approximately 30 and 120 milliseconds.

13. The method of claim 1, further comprising detecting an arrhythmia of the heart based on the electrical activity.

14. The method of claim 1, wherein sensing electrical activity of the chamber comprises sensing an evoked response resulting from delivery of excitatory extra-systolic electrical stimulation.

15. The method of claim 1, further comprising delivering pacing stimulation to the chamber via the first set of the electrodes.

16. A medical device system comprising:
first and second sets of electrodes; and
a medical device coupled to the first and second sets of electrodes, wherein the medical device delivers excitatory extra-systolic electrical stimulation to a chamber of a heart of a patient via the first set of electrodes and senses electrical activity of the chamber via the second set of electrodes.

17. The system of claim 16, wherein the electrodes of the first and second sets of electrodes are adapted to be chronically implanted within the patient.

18. The system of claim 16, wherein the electrodes of the first and second sets of electrodes are adapted to be chronically electrically coupled to the heart.

19. The system of claim 16, wherein the first and second sets of electrodes are first and second pairs of electrodes.

20. The system of claim 16, wherein the first set of electrode comprises a first tip electrode and a first ring electrode, and the second set of electrodes includes at least two of the following: the first ring electrode, a second tip electrode, a second ring electrode, a coil electrode, a subcutaneous electrode, a can-based electrode, a surface electrode, a pericardial electrode, an epicardial electrode, an endocardial electrode, a cardiac vein-based electrode.

21. The system of claim 16, further comprising a lead, wherein the electrodes of the first and second set of electrodes are operatively coupled to the lead.

22. The system of claim 16, further comprising first and second leads, wherein the electrodes of the first set of electrodes operatively coupled to the first lead, the electrodes of the second set of electrodes are operatively coupled to the second lead, and the first and second leads adapted to extend into the chamber.

23. The system of claim 22, wherein the chamber is the right ventricle, at least one of the electrodes of the first set of electrodes is adapted to be chronically located proximate to one of a ventricular outflow tract and a ventricular septum, and at least one of the electrodes of the second set of electrodes is adapted to be chronically located proximate to an apical portion of the heart.

24. The system of claim 16, wherein the chamber is the right ventricle.

25. The system of claim 16, wherein the medical device comprises a sense amplifier coupled to the second set of electrodes to sense electrical activity within the heart, and applies a far-field blanking interval to the sense amplifier in response to delivery of excitatory extra-systolic electrical stimulation via the first set of electrodes.

26. The system of claim 25, wherein a length of the far-field blanking interval is less than approximately 300 milliseconds.

27. The system of claim 25, wherein the length of the far-field blanking interval is between approximately 30 and 120 milliseconds.

28. The system of claim 25, wherein the medical device decouples the sense amplifier from the second set of electrodes during the blanking interval.

29. The system of claim 25, wherein the sense amplifier is selectively coupled to the second set of electrodes by a switch matrix.

30. The system of claim 16, wherein the medical device detects an arrhythmia of the heart based on the electrical activity sensed within the heart.

31. The system of claim 16, wherein the medical device senses an evoked response resulting from delivery of excitatory extra-systolic stimulation via the second set of electrodes.

32. The system of claim 16, wherein the medical device comprises a cardiac pacemaker, and delivers pacing stimulation via the first set of electrodes.

33. The system of claim 16, wherein the medical device is adapted to be chronically implanted within the patient.

34. A medical device system comprising:
an implantable pacemaker adapted to be implanted within a patient;
first and second leads each having a proximal end coupled to the pacemaker and a distal end adapted to operatively couple to a chamber of a heart of the patient;
a first pair of electrodes disposed on the distal end of the first lead; and
a second pair of electrodes disposed on the distal end of the second lead,
wherein the pacemaker delivers excitatory extra-systolic stimulation to the chamber via the first pair of electrodes, and senses electrical activity of the chamber via the second pair of electrodes.

35. The system of claim 34, wherein the first pair of electrodes comprises a first tip electrode and a first ring electrode, and the second pair of electrodes comprises at least two of the following: a second tip electrode, a second ring electrode, a coil electrode, a can-based electrode, an electrode coupled to the second lead, a subcutaneous electrode, a surface electrode, a pericardial electrode, an epicardial electrode, an endocardial electrode, a cardiac vein-based electrode.

36. The system of claim 35, wherein the chamber is a right ventricle of the heart, the first tip electrode is adapted to be chronically located proximate to one of a ventricular outflow tract and a ventricular septum of the heart, and the second tip electrode is adapted to be chronically located proximate to an apex of the heart.

37. A computer readable medium for storing executable instructions for performing a method, comprising:
instructions for delivering excitatory extra-systolic electrical stimulation to a chamber of a heart of a patient via a first set of electrodes; and
instructions for sensing electrical activity of the chamber via a second set of electrodes.

38. The method of claim 37, wherein the electrodes of the first and second sets of electrodes are adapted to be chronically implanted within the patient.

39. A medium according to claim 38, wherein the electrodes of the first and second sets of electrodes are adapted to be chronically located within the heart.

40. A medium according to claim 37, wherein the first and second sets of electrodes are first and second pairs of electrodes.

41. A medium according to claim 37, wherein the first set of electrodes includes a first tip electrode and a first ring electrode, and the second set of electrodes includes at least one of the first ring electrode, a second tip electrode, and second ring electrode, a coil electrode, a can electrode, a pericardial electrode, an epicardial electrode, an endocardial electrode.

42. A medium according to claim 37, wherein the electrodes of the first and second set of electrodes are operatively coupled to a single medical electrical lead.

43. A medium according to claim 37, wherein the electrodes of the first set of electrodes are operatively coupled to a first lead, the electrodes of the second set of electrodes are operatively coupled to a second lead, and the first and second leads are adapted to extend into the chamber.

44. A medium according to claim 37, wherein the instructions for delivering excitatory extra-systolic electrical stimulation to a chamber further comprises instructions for delivering excitatory extra-systolic electrical stimulation to a right ventricle of the heart, and instructions for sensing electrical activity within the chamber comprises sensing electrical activity within the right ventricle.

45. A medium according to claim 44, wherein the instructions for delivering excitatory extra-systolic electrical stimulation to a right ventricle further comprises instructions for delivering excitatory extra-systolic electrical stimulation to one of a site proximate to an outflow tract of the heart and a ventricular septum of the heart, and wherein the instructions for sensing electrical activity within the right ventricle comprises instructions for sensing electrical activity from a site proximate to an apex of the heart.

46. A medium according to claim 37, wherein the instructions for sensing electrical activity within the heart further comprises instructions for applying a far-field blanking interval to a sense amplifier coupled to the second set of electrodes in response to delivery of excitatory extra-systolic electrical stimulation via the first set of electrodes.

47. A medium according to claim 46, wherein a length of the far-field blanking interval is less than approximately 300 milliseconds

48. A medium according to claim 46, wherein the length of the far-field blanking interval is between approximately 30 and 120 milliseconds.

49. A medium according to claim 37, further comprising instructions for detecting an arrhythmia of the heart based on the electrical activity.

50. A medium according to claim 37, wherein the instructions for sensing electrical activity of the chamber comprises instructions for sensing an evoked response resulting from delivery of excitatory extra-systolic electrical stimulation.

51. A medium according to claim 37, further comprising instructions for delivering pacing stimulation to the chamber via the first set of the electrodes.
